(19) United States
(12) Patent Application Publication
(10) Pub. No.: US 2026/0076021 A1
(43) Pub. Date: Mar. 12, 2026

(54) EMITTER MATERIAL FOR OLEDS

(71) Applicant: TECHNISCHE UNIVERSITÄT DORTMUND, Dortmund (DE)

(72) Inventors: Andreas STEFFEN, Paderborn (DE); Thomas FERSCHKE, Würzburg (DE); André MUTHIG, Dortmund (DE); Jens PFLAUM, Dettelbach (DE); Ewald BJÖRN, Würzburg (DE)

(21) Appl. No.: 19/108,296

(22) PCT Filed: Aug. 29, 2022
(86) PCT No.: PCT/EP2022/073938

§ 371 Date: Mar. 3, 2025

(51) Int. Cl. H10K 50/11; C07F 1/08; C09K 11/06; H10K 50/86; H10K 85/30

(52) U.S. Cl. CPC H10K 50/11; C07F 1/08; C09K 11/06; H10K 50/86; H10K 85/341; C09K 2211/188

(57) ABSTRACT

The present disclosure relates to emitter materials for OLEDs based on defined metal complexes and their corresponding application in OLEDs.

The present disclosure relates to an emitter layer for an OLED which, for example, emits circularly polarised light, the emitter layer having a defined metal complex. The present disclosure further relates to an OLED comprising such an emitter layer, a method for generating light and the use of a metal complex for generating light in an emitter layer of an OLED. In addition, the present disclosure comprises a ligand for a corresponding metal complex.

OLEDs are known today for a variety of applications. In particular, they offer advantages for TVs or displays, and for display devices in general, due to their low-energy operation and simultaneously good image quality with high contrast.

Displays based on OLEDs are equipped with filters that reduce the reflection of other light sources, such as daylight, and thus prevent a loss of contrast. Linear polarisers combined with quarter-wave plates are used as filters, although only around 50% of the light emitted by the emitter layer can pass through and be detected by the user. Therefore, a high electrical power is typically required for a corresponding brightness to compensate for the light loss. In contrast, the circularly polarised light (circularly polarised luminescence, CPL) generated by chiral emitter materials with a high dissymmetry factor gₗᵤₘ can pass through the filters 100%, whereby a significantly higher energy efficiency can be achieved. However, these are not yet sufficiently known in the state of the art.

Efficient commercial OLEDs currently typically require phosphorescent triplet emitters, usually in the form of metal complexes of the expensive elements, iridium or platinum, to efficiently convert all excitons formed by electron-hole recombination into light. An alternative is luminescence via thermally activated delayed fluorescence (TADF), which can be realised with complexes of the much cheaper element copper or cheap organic compounds. While green-emitting OLEDs perform well, efficient electroluminescence in the deep red colour range is still a challenge.

### SUMMARY

It is thus an object of the present disclosure to overcome at least one disadvantage of the prior art, at least in part. In particular, it is an object of the present disclosure to provide an emitter layer that enables efficient electroluminescence in the deep red colour range.

A paracyclophane-carbene compound is described, characterised in that the paracyclophane-carbene compound corresponds to the following structure (I):

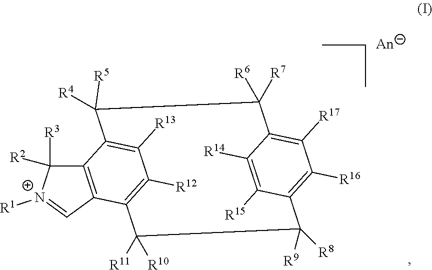

wherein
R¹ is selected from aryl, heteroaryl, alkyl, perfluoroalkyl, perfluoroaryl, wherein
R² and R³ are the same or different and are independently selected from aryl, heteroaryl, alkyl, alkenyl, alkynyl, perfluoroalkyl, perfluoroaryl, wherein
R⁴, R⁵, R⁶, R⁷, R⁸, R⁹, R¹⁰, R¹¹ are the same or different and are independently selected from hydrogen, deuterium, halogen, silyl, alkyl, alkenyl, alkynyl, perfluoroalkyl, aryl, heteroaryl, alkoxy, wherein
R¹² R¹³, R¹⁴, R¹⁵, R¹⁶, R¹⁷ are the same or different and are independently selected from hydrogen, deuterium, halogen, alkyl, alkenyl, alkynyl, perfluoroalkyl, aryl, heteroaryl, alkoxy, COR¹⁸, CO₂R¹⁹, CONR²⁰, cyano, nitro, SO₃R²¹, PO₃R²²₂, wherein
R¹⁸ R¹⁹, R²⁰, R²¹, R²² are the same or different and are independently selected from hydrogen, deuterium, halogen, silyl, alkyl, alkenyl, alkynyl, perfluoroalkyl, aryl, heteroaryl, alkoxy, and wherein
An⁻ is an anion.

The compound shown in structure (I) is shown as a salt, where the cation serves as a ligand that can form a bond to a metal. In this respect, it will be immediately apparent to those skilled in the art that, when reference is made to a corresponding ligand in the context of the present disclosure, this refers to the paracyclophane carben cation, which can then be bonded, or is bonded, to a metal atom as is usual for ligands.

In principle, for example, a C1 alkyl should denote an alkyl radical with one carbon atom, wherein the corresponding designations for the other radicals are to be understood accordingly, so that, for example, a C12 alkoxy denotes an alkoxy group with 12 carbon atoms.

Furthermore, the term An⁻ basically describes an anion regardless of its valency. The paracyclophane-carbene cation and the anion are intended to form a neutral salt. Depending on the selected anion, there may thus be, for example, one, two or more cations in order to form the paracyclophane-carbene compound according to structure (I) as a neutral salt overall.

A paracyclophane-carbene compound according to structure (I) can be particularly advantageous for forming a ligand for a metal complex that serves as an emitter material in an OLED.

In terms of use in an OLED, luminescence via thermally activated delayed fluorescence (TADF) represents an alternative to solutions from the state of the art, which can be realised with complexes of very cheap metals, such as copper, or with cheap organic compounds. While green-emitting OLEDs perform well, efficient electroluminescence in the deep red colour range is still a challenge.

In contrast to established iridium emitters, the ligand according to the disclosure is used to turn commercially available starting materials into racemic metal complexes, such as copper(I) complexes, which can be produced very cheaply and which, due to TADF, exhibit the highest radiation constants reported to date for molecular emitters in the red range of up to 2×10⁶ s⁻¹ and have also been successfully applied in a test OLED.

When used as ligands of metal complexes in emitter materials for OLEDs, the ligands described can thus enable a very high light yield and thus can significantly increase the quality of OLEDs. Furthermore, they can be operated with a comparatively very low energy input.

Due to their chiral nature, these compounds are also suitable in enantiopure form for use in OLEDs based on circularly polarised luminescence (CPL) in order to pass the anti-reflective filters of the displays with maximum efficiency. This new class of emitters thus shows the potential to be of interest for commercial energy-efficient applications. The positive properties can probably be further improved, if necessary, by further optimising the molecular structure as well as the OLED structure.

In principle, an emitter layer according to the present disclosure can thus be customised for the respective applications.

Furthermore, the possible synthetic routes can make the production significantly cheaper and/or easier compared to the iridium or platinum complexes used in the prior art, for example.

Thus, according to the disclosure, a low-energy operation of an OLED can be combined with a high light yield and thus a high quality while keeping costs moderate, wherein in particular a high light yield in the red spectral range is possible.

With respect to the paracyclophane-carbene compound that can serve as a ligand in a metal complex of an emitter layer of an OLED, it may be particularly preferred that
R¹ is selected from C5 to C18 aryl, C5 to C18 heteroaryl, C1 to C18 alkyl, C1 to C18 perfluoroalkyl, C5 to C18 perfluoroaryl, wherein
R² and R³ are the same or different and are independently selected from C5 to C18 aryl, C5 to C18 perfluoroaryl, C5 to C18 heteroaryl, C1 to C12 alkyl, C1 to C12 alkenyl, C1 to C12 alkynyl, C1 to C12 perfluoroalkyl, wherein
R⁴, R⁵, R⁶, R⁷, R⁸, R⁹, R¹⁰, R¹¹ are the same or different and are independently selected from hydrogen, deuterium, halogen, silyl, C1 to C12 alkyl, C1 to C12 alkenyl, C1 to C12 alkynyl and C1 to C12 perfluoroalkyl, C5 to C18 aryl, C5 to C18 perfluoroaryl, C5 to C18 heteroaryl, C1 to C12 alkoxy, wherein
R¹² R¹³, R¹⁴, R¹⁵, R¹⁶, R¹⁷ are the same or different and are independently selected from hydrogen, C1 to C18 alkyl, C1 to C18 alkenyl, C1 to C18 alkynyl, and C1 to C18 perfluoroalkyl, C5 to C18 aryl, C5 to C18 perfluoroaryl, C5 to C18 heteroaryl, C1 to C18 alkoxy, COR¹⁸, CO₂R¹⁹, CONR²⁰, cyano, nitro, SO₃R²¹, PO₃R²²₂, wherein
R¹⁸ R¹⁹, R²⁰, R²¹, R²² are the same or different and are independently selected from hydrogen, deuterium, halogen, silyl, C1 to C18 alkyl, C1 to C18 alkenyl, C1 to C18 alkynyl, C1 to C18 perfluoroalkyl, C5 to C18 aryl, C5 to C18 heteroaryl, C1 to C18 alkoxy, and wherein
An⁻ is an anion selected from halide, triflate, sulphate, phosphate, silicate, tetrafluoroborate, hexafluorophosphate, tetraarylborate.

More preferably, per an embodiment, R¹ may be selected from C9 to C18 aryl, C9 to C18 heteroaryl, C1 to C12 alkyl, C1 to C12 perfluoroalkyl, C9 to C18 perfluoroaryl, wherein R² and R³ are the same or different and are independently selected from C5 to C6 aryl and C5 to C6 heteroaryl,
R⁴, R⁵, R⁶, R⁷, R⁸, R⁹, R¹⁰, R¹¹ are the same or different and are independently selected from hydrogen, deuterium, halogen and silyl, C1 to C4 alkyl, C1 to C4 alkenyl, C1 to C4 alkinyl and C1 to C4 perfluoroalkyl, C5 to C6 aryl, C5 to C6 heteroaryl, C5 to C6 perfluoroaryl, C1 to C4 alkoxy, wherein
R¹² R¹³, R¹⁴, R¹⁵, R¹⁶, R¹⁷ are the same or different and are independently selected from hydrogen, C1 to C4 alkyl, C1 to C4 alkenyl, C1 to C4 alkynyl, and C1 to C4 perfluoroalkyl, C5 to C6 aryl, C5 to C6 perfluoroaryl, C5 to C6 heteroaryl, C1 to C4 alkoxy, COR¹⁸, CO₂R¹⁹, CONR²⁰, cyano, nitro, SO₃R²¹, PO₃R²²₂, wherein
R¹⁸ R¹⁹, R²⁰, R²¹, R²² are the same or different and are independently selected from hydrogen, deuterium, halogen, silyl, C1 to C4 alkyl, C1 to C4 alkenyl, C1 to C4 alkynyl, C1 to C4 perfluoroalkyl, C5 to C12 aryl, C5 to C12 heteroaryl, C1 to C4 alkoxy, and wherein
An⁻ is an anion selected from halide, triflate, sulphate, phosphate, silicate, tetrafluoroborate, hexafluorophosphate, tetraarylborate.

In particular, R¹ may be a dialkyl phenyl. For example, the dialkyl phenyl may be 2,6-diisopropylphenyl.

It may also be preferred, per an embodiment, that R² and R³ are phenyl. In principle, the phenyl rings may be substituted, but it is also possible that the phenyl rings are unsubstituted.

It may further be preferred, per an embodiment, that R⁴, R⁵, R⁶, R⁷, R⁸, R⁹, R¹⁰ and R¹¹ are hydrogen.

In addition or in the alternative, per an embodiment, it may be advantageous that R¹² R¹³, R¹⁴, R¹⁵, R¹⁶ and R¹⁷ are hydrogen.

In a specific embodiment of the present disclosure, it may be provided that the paracyclophane-carbene compound has the following structure (II):

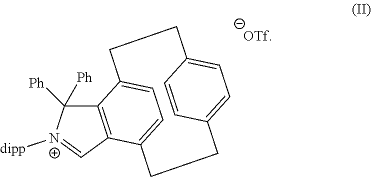

In this structure, the group ‘dipp’ refers to 2,6-diisopropylphenyl and the anion OTF-refers to a triflate group, which may also be referred to as a trifluoromethanesulfonate group.

In accordance with the above, the present disclosure further relates to a use of a paracyclophane-carbene compound as described above for producing a metal complex for an emitter material of an emitter layer of an organic light-emitting diode.

In summary, as described in detail above with regard to the paracyclophane-carbene compound, a low-energy operation of an OLED can be combined with a high light yield and thus high quality while keeping costs moderate, wherein in particular a high light yield in the red spectral range is possible.

With regard to further advantages and technical features of the paracyclophane-carbene compound and its use, reference is made to the description of the metal complex, its use as an emitter material in an emitter layer, the emitter layer, the organic light-emitting diode, the method, the figures and the description of the figures.

Furthermore, a metal complex is described. The metal complex is characterised by having the following structure (III):

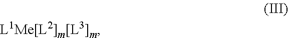

wherein L¹ is a paracyclophane-carbene ligand as described above, wherein
L² and L³ are the same or different and are independently selected from
NR²³₂, NR²⁴₃, PR²⁵₂, PR²⁶₃, OR²⁷₂, OR²⁸, SR²⁹, SR³⁰₂, SR³¹, SeR³²₂, SeR³⁴, TeR³⁵₂, TeR³⁶, silyl, aryl, heteroaryl, alkenyl, alkynyl, isonitrile, cyclopentadienyl, halogen, wherein m is a number selected from 0, 1 or 2, with the proviso that at least one m is not equal to zero, wherein
R²³, R²⁴, R²⁵, R²⁶, R²⁷, R²⁸, R²⁹, R³⁰, R³¹, R³², R³³, R³⁴, R³⁵, R³⁶ are the same or different and are independently selected from aryl, heteroaryl, alkenyl, alkynyl, alkyl, perfluoroalkyl, perfluoroaryl; and wherein
Me is a metal selected from copper, silver and gold.

With regard to the description of the ligand L¹ as a paracyclophane-carbene ligand, the following should be noted. The paracyclophane-carbene-based salt is reacted with a base and the resulting neutral carbene ligand is bound to the metal, as is comprehensively described for carbenes in the literature and is generally known. In other words, the paracyclophane-carbene compound can also be described as a corresponding ligand salt with a paracyclophane-carbene structural unit as the ligand.

In principle, only one ligand L2 can be present in addition to the ligand L1 on the metal of the metal complex, but no ligand L3, only one ligand L3 but no ligand L2, or both ligands L2 and L3 can be present. Furthermore, the ligands L2 and L3 can be separated from each other or there can be a ligand L2 which binds to the metal with two bonds and can thus be designated as a bridging ligand or chelate ligand.

Such a metal complex is used in particular as an emitter material in an emitter layer of an organic light-emitting diode. This has the particular advantage, per an embodiment, of enabling efficient electroluminescence, especially in the deep red colour range. This is often still a challenge with solutions from the state of the art.

In contrast to established iridium emitters, the metal complex according to the disclosure describes racemic metal complexes that can be produced very cheaply from commercial starting materials and that, due to TADF, have the highest radiation constants reported to date for molecular emitters in the red range of up to 2×10⁶ s⁻¹ and have also been successfully applied in a test OLED.

In summary, a very high light yield can be achieved and thus the quality of OLEDs can be significantly increased. Furthermore, they can be operated with a comparatively very low energy input.

Thus, according to the disclosure, a low-energy operation of an OLED can be combined with a high light yield and thus a high quality while keeping costs moderate, wherein in particular a high light yield in the red spectral range is possible.

It may be preferred that
L¹ is a paracyclophane-carbene ligand as described above, wherein
Me is a metal selected from copper, silver and gold, and wherein
L² is a ligand selected from a heteroaryl and a halogen.

For example, it may be preferred that
L¹ is a paracyclophane-carbene ligand as described above, wherein
Me is copper, and wherein
L² is a ligand selected from a carbazole ligand and a halogen.

In particular, the use of a copper complex, for example, allows for the formation of cost-effective complexes in comparison to iridium complexes from the prior art. Accordingly, the advantages in this regard, per an embodiment, may be particularly pronounced.

In a particular embodiment, the metal complex may be selected from the structures (IV), (V), (VI), (VII), (VIII) and (IX) as shown below:

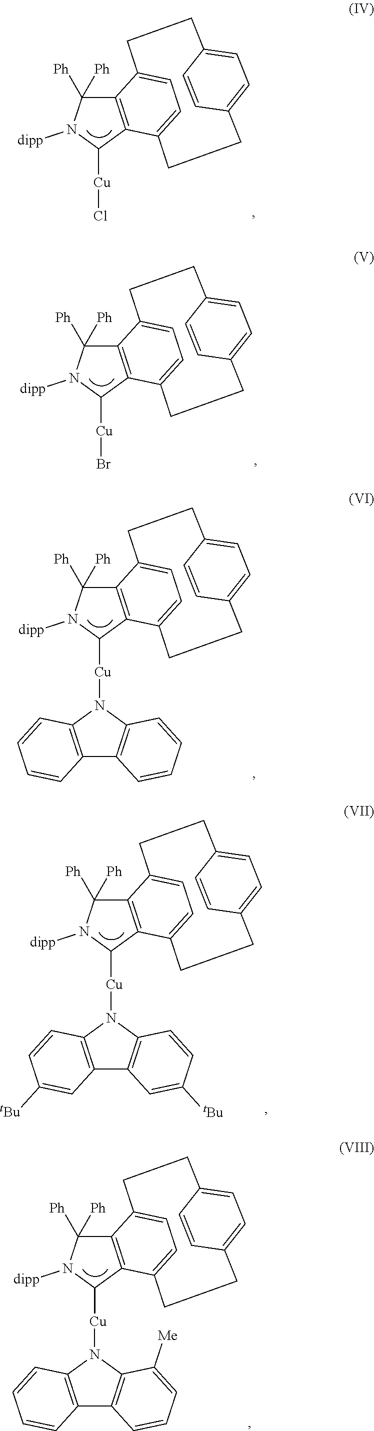

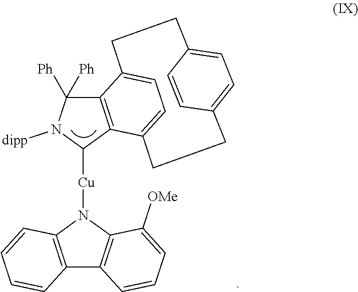

In accordance with the above, the present disclosure also relates to the use of a metal complex as described above as an emitter material in an emitter layer of an organic light-emitting diode.

In summary, as described above with regard to the paracyclophane-carbene compound and the corresponding metal complex, a low-energy operation of an OLED can be combined with a high light yield and thus high quality at moderate cost, wherein in particular a high light yield in the red spectral range is possible.

With regard to further advantages and technical features of the metal complex and its use as an emitter material in an emitter layer, reference is made to the description of the paracyclophane-carbene compound and its use, the emitter layer, the organic light-emitting diode, the method, the figures and the description of the figures.

Furthermore, an emitter layer for an organic light-emitting diode is described, comprising a metal complex for emitting light, characterised in that the metal complex is formed as described above.

An emitter layer for an organic light-emitting diode (OLED) is thus described. In principle, the OLED can be constructed as is known from the prior art. Thus, no special requirements need to be met in order to implement the emitter layer described above in an OLED.

The emitter layer is characterised by the fact that it contains a metal complex as the active emitter material, as described above. In a general form, the emitter layer contains a metal complex that corresponds to the following structure (III):

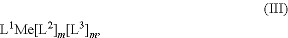

wherein L¹ is a paracyclophane-carbene ligand as described above, wherein
L² and L³ are the same or different and are independently selected from
NR²³₂, NR²⁴₃, PR²⁵₂, PR²⁶₃, OR²⁷₂, OR²⁸, SR²⁹, SR³⁰₂, SR³¹, SeR³²₂, SeR³⁴, TeR³⁵₂, TeR³⁶, silyl, aryl, heteroaryl, alkenyl, alkynyl, isonitrile, cyclopentadienyl, halogen, wherein
m is a number selected from 0, 1 or 2, with the proviso that at least one m is not equal to zero, wherein
R²³, R²⁴, R²⁵, R²⁶, R²⁷, R²⁸, R²⁹, R³⁰, R³¹, R³², R³³, R³⁴, R³⁵, R³⁶ are the same or different and are independently selected from aryl, heteroaryl, alkenyl, alkynyl, alkyl, perfluoroalkyl, perfluoroaryl;
and wherein
Me is a metal selected from copper, silver and gold.

In general, it may be preferred that
L¹ is a paracyclophane-carbene ligand as described above, wherein
Me is a metal selected from copper, silver and gold, and wherein
L² is a ligand selected from a heteroaryl and a halogen.

For example, it may be preferred that
L¹ is a paracyclophane-carbene ligand as described above, wherein
Me is copper, and wherein
L² is a ligand selected from a carbazole ligand and a halogen.

In particular, the use of a copper complex, for example, allows for the formation of cost-effective complexes in comparison to iridium complexes from the prior art. Accordingly, the advantages in this regard may be particularly pronounced, per an embodiment.

In a particular embodiment, the metal complex may be selected from the structures (IV), (V), (VI), (VII), (VIII) and (IX) as shown below:

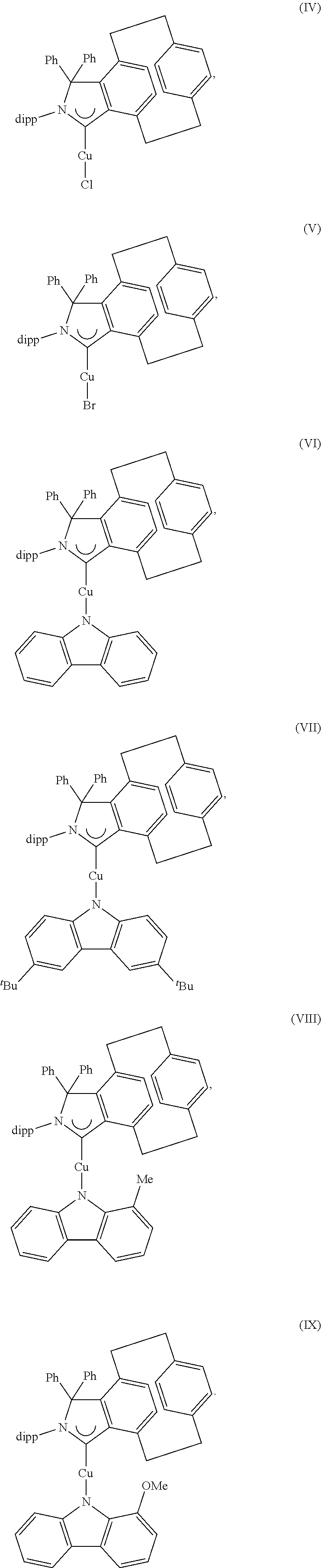

In summary, the invention describes ligands that can be used to produce racemic metal complexes, such as copper(I) complexes, very cheaply from commercial starting materials, in contrast to established iridium emitters, which, due to TADF, have the highest radiation constants in the red range of up to 2×10⁶ s⁻¹ reported to date for molecular emitters and have also been successfully applied in a test OLED.

When used as ligands of metal complexes in emitter materials for OLEDs, the ligands described can thus enable a very high light yield and thus significantly increase the quality of OLEDs. Furthermore, they can be operated with a comparatively very low energy input.

Thus, according to the disclosure, a low-energy operation of an OLED can be combined with a high light yield and thus a high quality while keeping costs moderate, wherein in particular a high light yield in the red spectral range is possible.

With regard to further advantages and technical features of the emitter layer, reference is made to the description of the paracyclophane-carbene compound and its use, the metal complex and its use as emitter material in an emitter layer, the organic light-emitting diode, the method, the figures and the description of the figures.

Furthermore, an organic light-emitting diode (OLED) is described, comprising a cathode, an emitter layer, a hole transport layer and an anode, characterised in that the emitter layer is formed as described above.

Accordingly, the metal complex forming the emitter material and located in the emitter layer can correspond to the following structure (III):

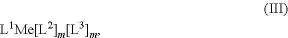

wherein L¹ is a paracyclophane-carbene ligand as described above, wherein
L² and L³ are the same or different and are independently selected from
NR²³₂, NR²⁴₃, PR²⁵₂, PR²⁶₃, OR²⁷₂, OR²⁸, SR²⁹, SR³⁰₂, SR³¹, SeR³²₂, SeR³⁴, TeR³⁵₂, TeR³⁶, silyl, aryl, heteroaryl, alkenyl, alkynyl, isonitrile, cyclopentadienyl, halogen, wherein
m⁻ is a number selected from 0, 1 or 2, with the proviso that at least one m is not equal to zero, wherein
R²³, R²⁴, R²⁵, R²⁶, R²⁷, R²⁸, R²⁹, R³⁰, R³¹, R³², R³³, R³⁴, R³⁵, R³⁶ are the same or different and are independently selected from aryl, heteroaryl, alkenyl, alkynyl, alkyl, perfluoroalkyl, perfluoroaryl; and wherein
Me is a metal selected from copper, silver and gold.

The metal complex of the emitter layer can be further developed, as described in detail above with reference to the description of the metal complex.

As described above in greater detail with regard to the emitter layer, the use of the copper complex as the emitter material in the emitter layer offers the advantages of low-energy operation of a suitably equipped OLED in combination with high luminous efficacy, particularly in the red spectral range, wherein low-cost producibility is also possible.

The structure of the OLED, comprising the cathode layer, emitter layer, hole transport layer and anode layer, can be formed in principle as is known from the state of the art.

For example, the cathode can have a metal or a metal alloy or consist of it, so that the cathode or its material has a low electron work function. Examples include calcium, aluminium, barium, ruthenium or a magnesium-silver alloy.

The anode can be, for example, a metallic oxide, such as indium tin oxide (ITO).

The hole transport layer (HTL) can be a molecular or polymeric material, such as poly(3,4-ethylenedioxythiophene) polystyrene sulfonate (PEDOT:PSS).

The emitter layer has as emitter material in particular the copper complex described above. This is preferably diluted, per an embodiment, with concentrations of, for example, 1-10 wt. %, preferably 1-5 wt. %, in a suitable matrix, wherein the proportions described above relate to the entire emitter layer. A suitable matrix material may be, for example, 1,3-bis(N-carbazolyl)benzene (mCP).

With regard to the matrix material, this can generally be selected from a wide range as long as the energy levels enable an energy transfer or direct recombination on the emitter. The exact material is to be selected in particular depending on the selected Cu complex energies, i.e. on the specifically selected ligands.

Furthermore, the layer structure described above can be applied to a substrate, such as a glass substrate.

The production and structure of the OLED can basically correspond to those of the prior art.

In principle, the OLED can only consist of the layers described above, although this does not exclude the possibility of further layers, which should be covered by the present disclosure. For example, optimised, doping charge transport layers or charge/exciton-blocking layers may be conceivable. The additional layers can, for example, be used for energy tailoring of the successive functional layers, such as balancing the carrier injection asymmetry, creating targeted energy cascades to optimise energy transfer to the emitter, etc.

It may be preferred that the organic light-emitting diode further comprises a filter for reducing reflections of external light sources, the filter being at least partially opaque to linearly polarised light.

In addition to the layers described above, the layer structure or the OLED thus preferably, per an embodiment, comprises a filter or a filter combination that serves to reduce reflections of external light sources. In particular, the filter combination consists of a linear polariser and a lambda/4 plate. A filter combination of this kind is characterised by the fact that it filters out at least some of the linearly polarised light from a beam path and thus filters out daylight from the beam path. While a part of the generated linearly polarised light is also blocked by this filter arrangement in the emission of standard OLEDs and does not contribute to light generation, with the proposed emitters, which in particular emit circularly polarised light, in principle each of the generated light particles can be decoupled and contribute to light generation. This significantly reduces reflections of daylight, while the internally generated light can pass completely through the filter arrangement and thus significantly improve the contrast of an image emitted by an OLED, particularly under traditionally difficult conditions, such as strong sunlight outdoors.

In particular, such a filter can provide advantages for an OLED as described above, since an OLED provided with a linear polariser as a filter does not reduce the light yield of circularly polarised light. Therefore, a high-quality image can be achieved in particular with an OLED according to the disclosure.

With regard to further advantages and technical features of the organic light-emitting diode, reference is made to the description of the paracyclophane-carbene compound and its use, the metal complex and its use as the emitter material in an emitter layer, the emitter layer, the method, the figures and the description of the figures.

Furthermore, a method for generating in particular circularly polarised light by means of an organic light-emitting diode is described, comprising the method steps:
a) providing an organic light-emitting diode as described above; and
b) applying a voltage between the anode and the cathode for injecting charge carriers and for generating in particular circularly polarised light.

With regard to process step a) and in particular with regard to the formation and further development of the OLED and in particular of the emitter material in the emitter layer, full reference is made to the corresponding description of the OLED and the emitter layer.

By applying a voltage between the anode and cathode, which is feasible within the scope of what is known to a person skilled in OLEDs, the emitter material in the emitter layer can be excited to emit circularly polarised light in particular. This is possible in a surprising way with the copper complex described above, with high light yield, which brings significant advantages.

The OLED can be operated in a voltage range of a few volts and a current density range of up to 1 A/cm², for example at 10 mA/cm², which is common for established OLED architectures. This means that the OLED with the circularly emitting active layer in particular can be controlled by established electrical circuits and implemented in existing structures (e.g. display units).

In summary, it can be permitted that images can be produced using the metal complexes described above as emitter material with high light efficiency, in particular in the red spectral range, and good contrast, whereby energy-efficient operation is also possible.

With regard to further advantages and technical features of the process, reference is made to the description of the paracyclophane-carbene compound and its use, the metal complex and its use as emitter material in an emitter layer, the emitter layer, the organic light-emitting diode, the figures and the description of the figures.

### BRIEF DESCRIPTION OF THE FIGURES

The invention is explained below with reference to the accompanying drawings and examples, wherein the features presented below may each individually and in combination represent an aspect of the invention, and wherein the invention is not limited to the following drawing, the following description and the following example.

The following show:

FIG. 1 a schematic representation of a structure of an OLED in a configuration according to an embodiment;

FIG. 2 a schematic representation of a structure of an OLED in a further configuration according to an embodiment;

FIG. 3 a reaction scheme for producing the paracyclophane-carbene compound according to an embodiment;

FIG. 4 a ¹H-NMR spectrum of the compound 6;

FIG. 5 a ¹³C-NMR spectrum of compound 6;

FIG. 6 a ¹H-NMR spectrum of compound 7;

FIG. 7 a ¹³C-NMR spectrum of compound 7;

FIG. 8 a ¹H-NMR spectrum of compound 9;

FIG. 9 a ¹³C-NMR spectrum of compound 9;

FIG. 10 a ¹H-NMR spectrum of compound 10;

FIG. 11 a ¹³C-NMR spectrum of compound 10;

FIG. 12 a ¹H-NMR spectrum of compound 11;

FIG. 13 a ¹³C-NMR spectrum of compound 11;

FIG. 14 a ¹H-NMR spectrum of compound 12;

FIG. 15 a ¹³C-NMR spectrum of compound 12;

FIG. 16 normalised excitation and emission spectra of compounds 6, 7 and 8;

FIG. 17 normalised excitation and emission spectra of compounds 9, 10, 11 and 12;

FIG. 18 normalised excitation and emission spectra of compounds 9, 10, 11 and 12;

FIG. 19 an EL spectrum of an exemplary OLED; and

FIG. 20 the current-voltage characteristic at positive voltage of an exemplary OLED.

### DETAILED DESCRIPTION

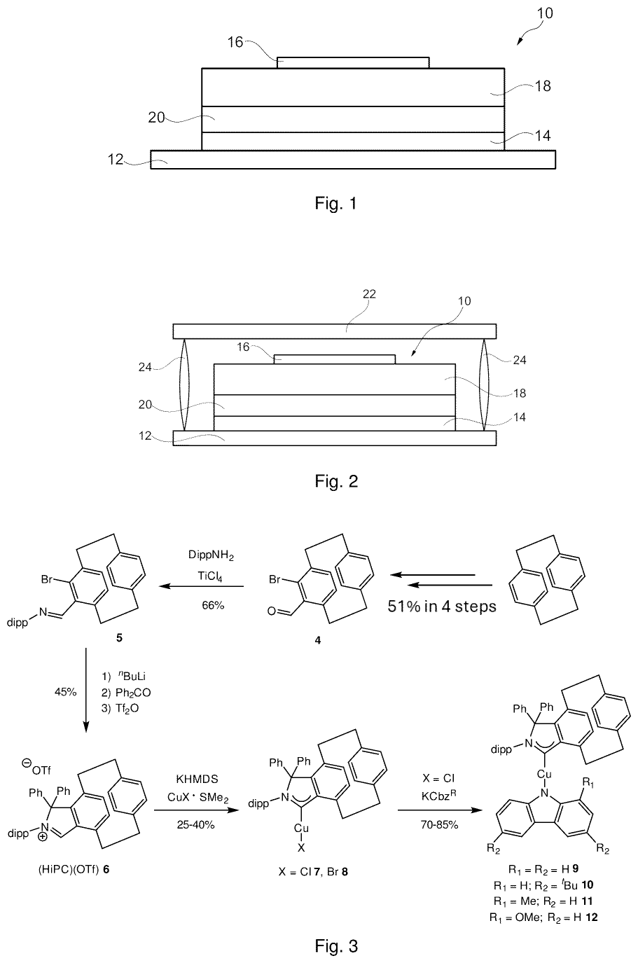

FIG. 1 shows a configuration of a layer structure 10 for an OLED according to a configuration of the present disclosure. The layer structure 10 comprises an anode 14, a cathode 16 and an emitter layer 18. The anode 14 may comprise, for example, consist of a material with a moderate work function, such as ITO, and the cathode 16 may comprise, for example, consist of a material with a low work function, such as calcium. The emitter layer 18 comprises a metal complex as described below.

Furthermore, a layer 20 is shown, which is a hole transport layer. This can be realised, for example, by the hole-transporting polymer poly(3,4-ethylenedioxythiophene) polystyrene sulfonate (PEDOT:PSS).

In principle, further layers may be present without departing from the scope of the disclosure. For example, a hole transport layer can be provided between the emitter layer 18 and the hole conduction layer 20, and/or an electron transport layer can be present between the emitter layer 18 and the cathode 16. In principle, the transport layers can also act as exciton and charge carrier blocking layers, or, due to their energetics, fulfil several functions in one.

FIG. 2 also shows a cover layer 22, such as a cover glass, which delimits the layer structure 10 at the top, with a seal 24 being shown between the cover layer 22 and the substrate 12, which may be formed from an adhesive, such as an epoxy adhesive. The substrate 12, the covering layer 22 and the seal can thus encapsulate the layer structure 10 and thereby protect it from external influences. This can, for example, minimise the degradation of the calcium and the organic material. The encapsulation can, for example, contain a protective gas, such as nitrogen.

Common to the embodiments of FIGS. 1 and 2 is that the emitter layer 18 comprises a defined copper complex as the emitter material. It is envisaged that the metal complex corresponds to the following structure (III):

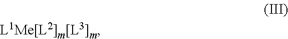

wherein L¹ is a paracyclophane-carbene ligand, wherein
L² and L³ are the same or different and are independently selected from
NR²³₂, NR²⁴₃, PR²⁵₂, PR²⁶₃, OR²⁷₂, OR²⁸, SR²⁹, SR³⁰₂, SR³¹, SeR³²₂, SeR³⁴, TeR³⁵₂, TeR³⁶, silyl, aryl, heteroaryl, alkenyl, alkynyl, isonitrile, cyclopentadienyl, halogen, wherein
m is a number selected from 0, 1 or 2, with the proviso that at least one m is not equal to zero, wherein
R²³, R²⁴, R²⁵, R²⁶, R²⁷, R²⁸, R²⁹, R³⁰, R³¹, R³², R³³, R³⁴, R³⁵, R³⁶ are the same or different and are independently selected from aryl, heteroaryl, alkenyl, alkynyl, alkyl, perfluoroalkyl, perfluoroaryl; and wherein
Me is a metal selected from copper, silver and gold.

By way of example, the metal complex may be selected from the structures (IV), (V), (VI), (VII), (VIII) and (IX) as shown below:

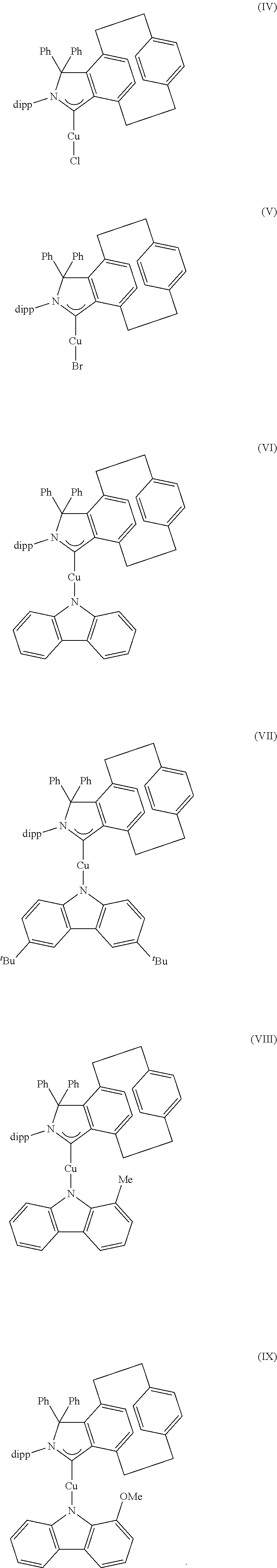

In the following, synthesis examples are shown for the emitter materials defined above, which are present in the emitter layer 18. Firstly, the synthesis of the ligands is discussed, in particular the ligand L¹. Subsequently, the synthesis of various metal complexes is described.

As shown below, structure (II) corresponds to compound 6, structure (IV) to compound 7, structure (VI) to compound 9, structure (VII) to compound 10, structure (VIII) to compound 11, structure (IX) to compound 12, structure (IV) to compound 7, structure (IV) to compound 7.

### Examples of the Synthesis of the Ligand Contained in the Copper Complex

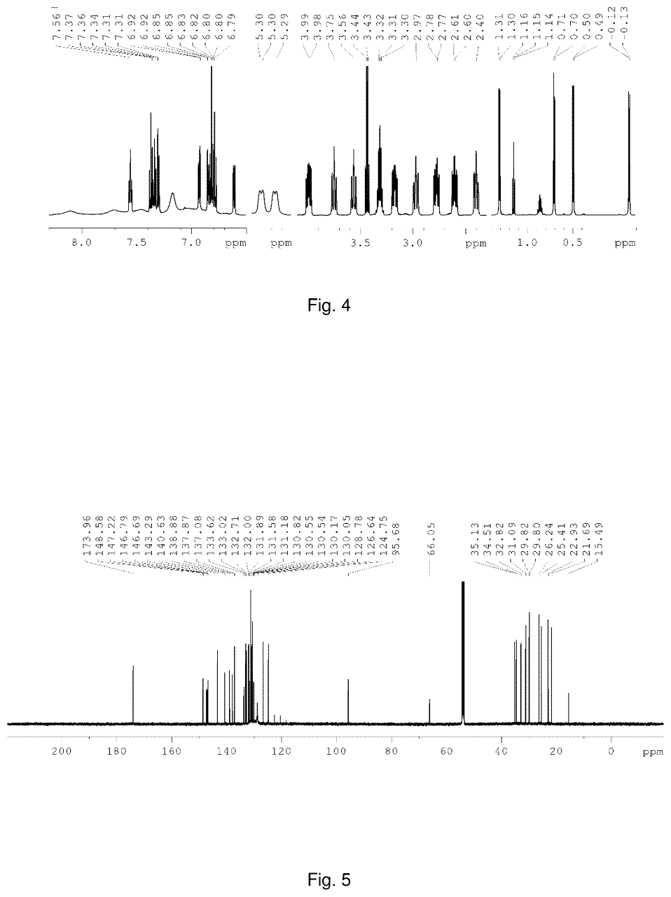

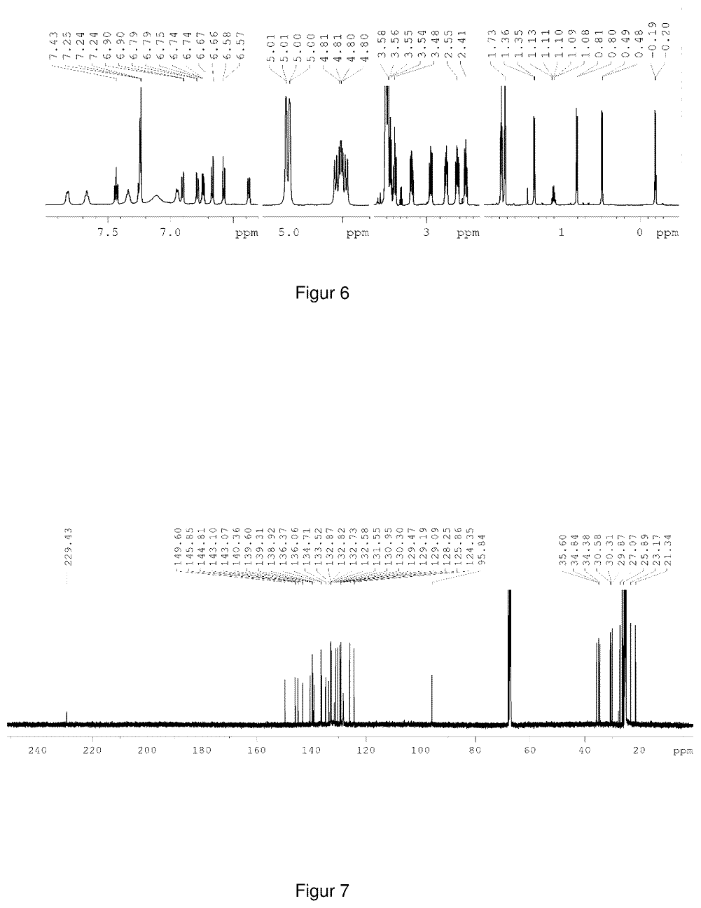

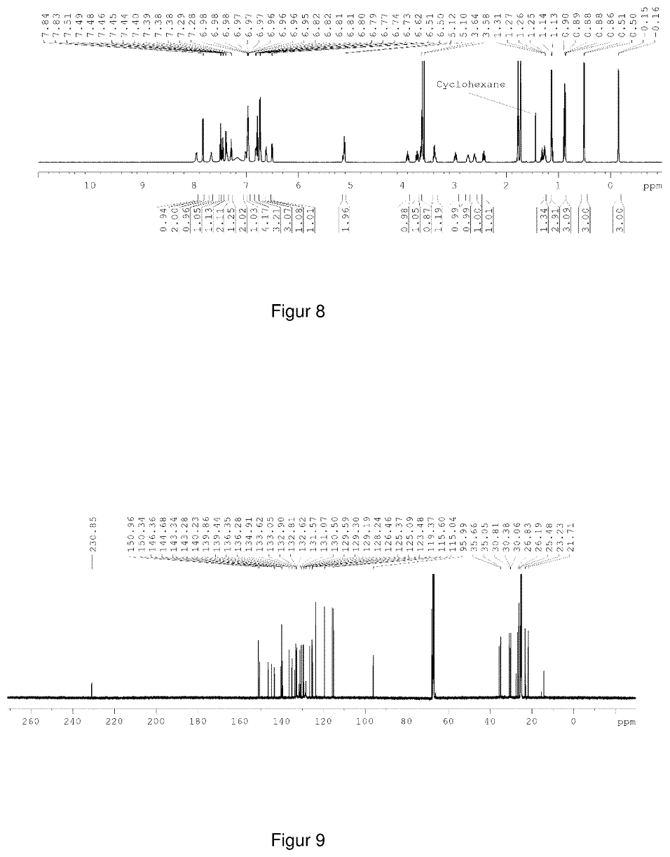

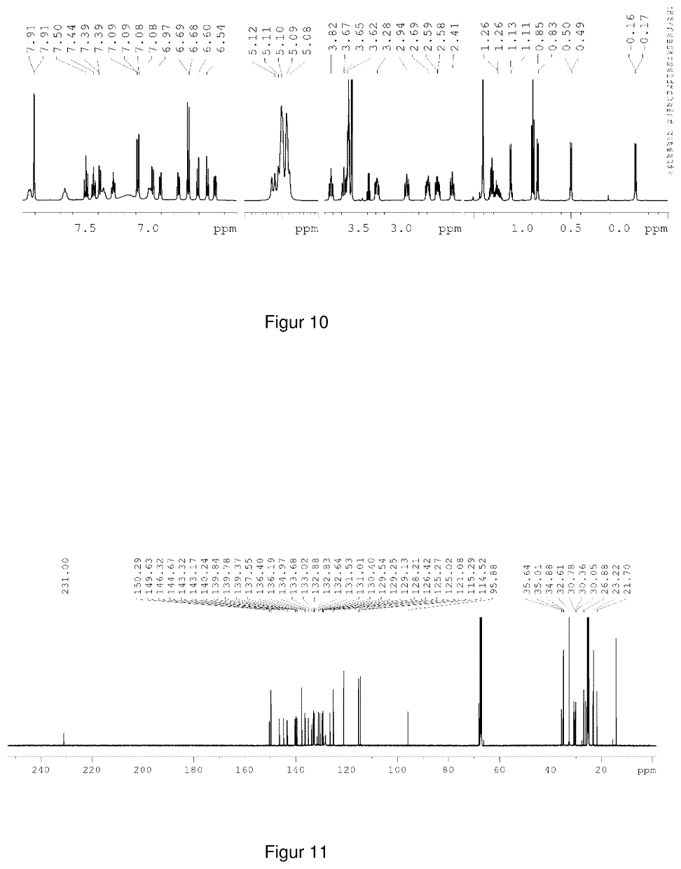

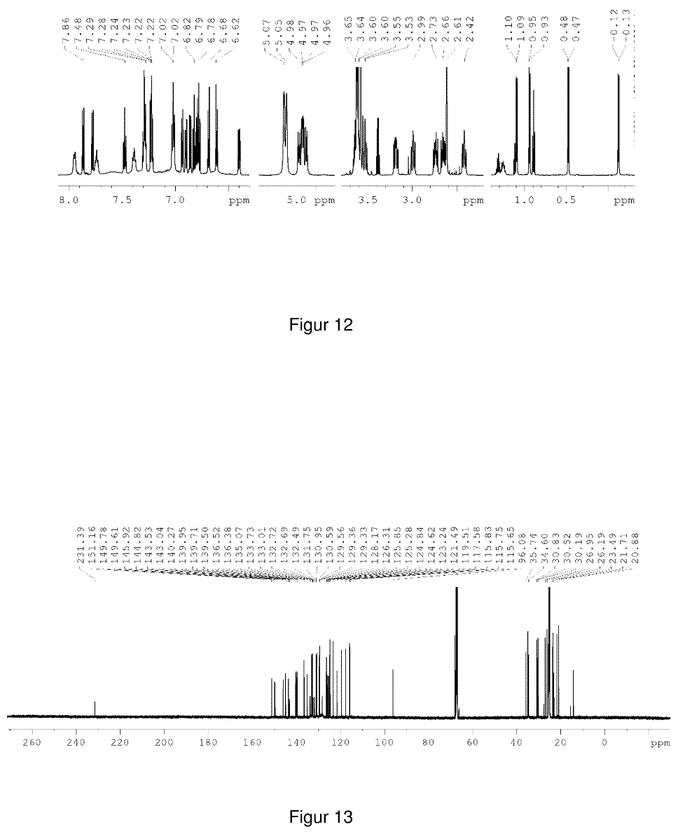

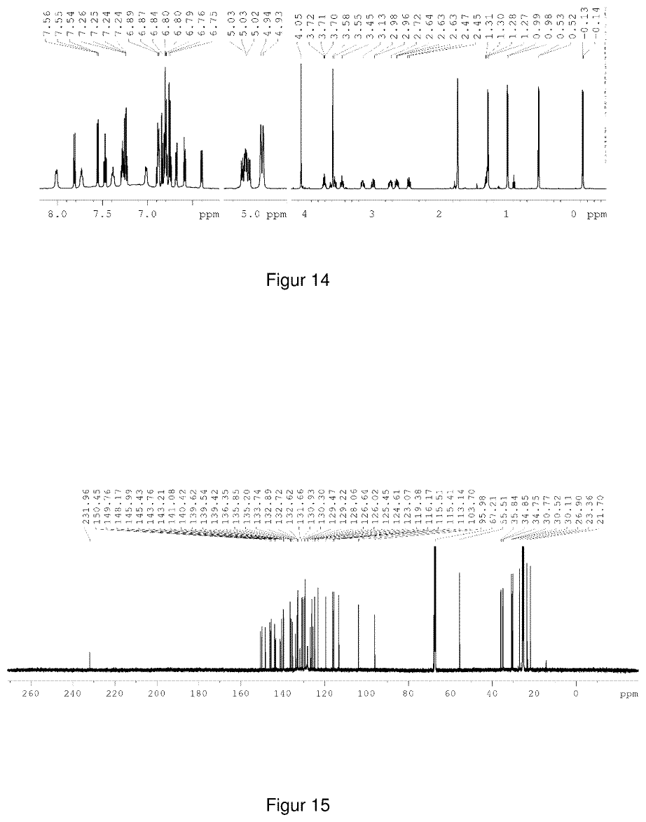

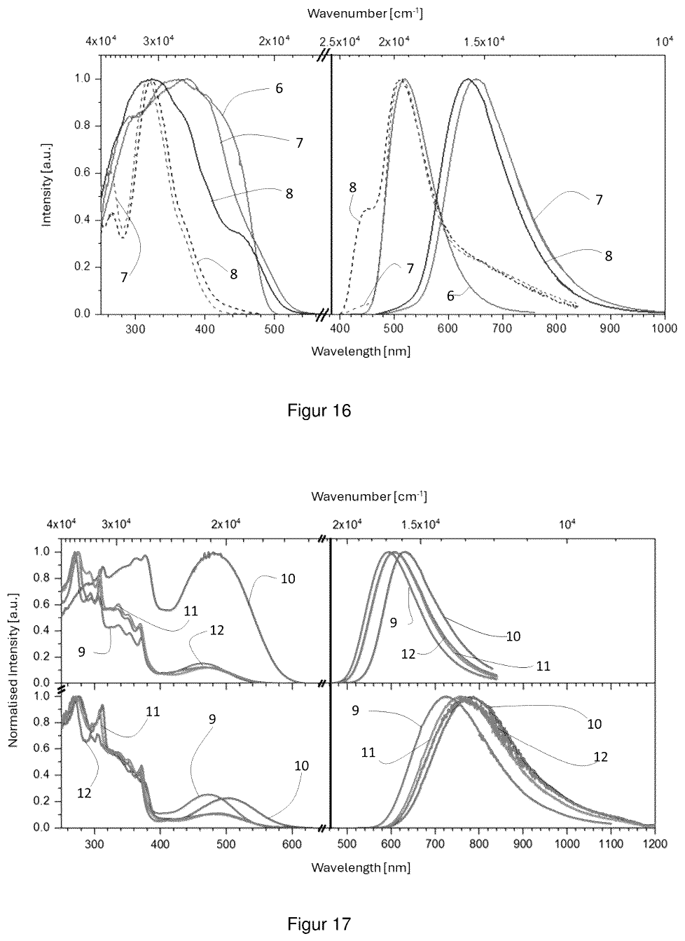

The paracyclophane-carbene ligand or the paracyclophane-carbene compound can be synthesised according to Scheme 1, which is shown as FIG. 3.

In detail, the synthesis proceeds as follows:

### rac-4-Formyl[2.2]paracyclophane (Compound 1)

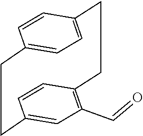 [SMILES: O=Cc1cc2ccc1CCc1ccc(cc1)CC2]

In a 500 mL Schlenk flask, 2.00 g (9.60 mmol, 1.0 eq) of -[2.2]paracyclophane in 150 mL DCM was cooled to 0° C. 0.90 ml (10 mmol, 1.05 eq) of titanium (IV) chloride and 2.1 ml (19 mmol, 2 eq) of dichloromethoxymethane were carefully added and stirred overnight. The black reaction solution was added to 200 ml of ice. The turquoise suspension was stirred for 2 h. The colourless organic phase was separated and the colourless aqueous phase was extracted twice with 50 ml DCM. The combined organic phase was dried with Na₂SO₄, filtered and all volatile components were removed under reduced pressure. The product was obtained as a colourless solid after washing with pentane (2.10 g, 8.89 mmol, 93%).

The spectroscopic data are known from the literature.

### rac-4-Formyl[2.2]paracyclophane-O-methylaldoxime (Compound 3)

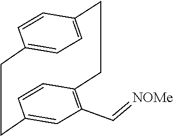

In a 100 mL Schlenk flask, 424 mg (5.08 mmol, 1.2 eq) of methoxyamine hydrochloride, 0.67 mL (8.4 mmol, 2 eq) of pyridine, and 0.5 g of molecular sieve (4 Å) were stirred in 12 mL of DCM for 5 min. 1.00 g (4.23 mmol, 1 eq) rac-4-formyl[2.2]paracyclophane was added and stirred overnight. The yellow reaction mixture was extracted three times with 20 ml DCM, filtered through silica and all volatile components were removed under reduced pressure. The product was obtained by column chromatography (silica; pentane:ethyl acetate 100:0→50:1) as a colourless solid 660 mg, 2.49 mmol, 59%).

The spectroscopic data are known from the literature.

### rac-4-Bromo-5-formyl[2.2]paracyclophane-O-methylaldoxime (Compound xx)

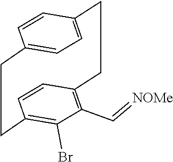

In a 100 mL Young tube, 200 mg (754 μmol, 1 eq) of rac-4-formyl[2.2]paracyclophane-O-methylaldoxime, 34 mg (0.15 mmol, 0.2 eq) of palladium (II) acetate, 33 mg (0.15 mmol, 0.2 eq) of silver (I) trifluoroacetate and 161 mg (905 μmol, 1.2 eq.) N-bromosuccinimide were stirred in 25 ml DCE for 6 h at 100° C. The reaction mixture was cooled to RT and 25 ml DCM and 20 ml dist. water were added. The aqueous phase was extracted twice with 20 ml DCM. The organic phase was dried with magnesium sulphate, filtered over Celite, and all volatile components were removed under reduced pressure. The crude product was used without further work-up.

The spectroscopic data are known from the literature.

### rac-4-Bromo-5-formyl[2.2]paracyclophane (Compound 4)

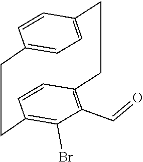 [SMILES: O=Cc1c2ccc(c1Br)CCc1ccc(cc1)CC2]

In a 10 ml microwave vial, 160 mg (465 μmol, 1 eq.) rac-4-bromo-5-formyl[2.2]paracyclophane-O-methylaldoxime, 180 mg (946 μmol, 2.0 eq.) para-toluenesulfonic acid monohydrate and 0.35 ml (4.7 mmol, 10 eq., 37% in water) formaldehyde were irradiated in 5 ml THF and 1 ml water at 70 W (120° C.) for 4 h with microwaves. The reaction mixture was cooled to RT and all volatile components were removed under reduced pressure. The product was obtained by column chromatography (silica; cyclohexane:ethyl acetate 100:0→50:1) as a colourless solid 140 mg, 0.444 mmol, 96%).

The spectroscopic data are known from the literature.

### rac-4-Bromo-5-formyl[2.2]paracyclophane-N-(2,6-diisopropylphenyl) methaniminium (Compound 5)

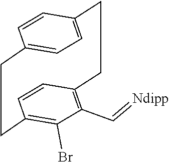

To a solution of 100 mg (317 μmol, 1.0 eq) of rac-4-bromo-5-formyl[2.2]paracyclophane and 0.60 mg (0.34 mmol, 1.1 eq) of N-(2,6-diisopropylphenyl)amine in 1 ml toluene, 0.07 ml (0.6 mmol, 2 eq.) titanium (IV) chloride was carefully added at 0° C. and the mixture was heated at reflux for 2 h. The orange reaction mixture was added to 3 ml isopropanol. 3 ml dist. water and 4 ml diethyl ether were added. The aqueous phase was extracted twice with 5 ml diethyl ether. The combined organic phase was dried with Na₂SO₄, decanted and all volatile components were removed under reduced pressure. The product was obtained by column chromatography (silica; cyclohexane:ethyl acetate 100:0→50:1) as a yellow oil 100 mg, 0.211 mmol, 66%).

The ¹H-NMR data are as follows: (500 MHz, CDCl₃) δ 8.26 (s, 1H), 7.21 (m, 2H), 7.15 m, 1H), 6.98 (m, 1H), 6.67 (m, 2H), 6.58 (m, 3H), 4.26 (m, 1H), 3.56 (m, 1H), 3.19 (m, 5H), 3.10 (m, 1H), 2.93 (m, 2H), 1.28 (d, J=6.8 Hz, 6H), 1.24 (d, J=6.9 Hz, 6H).

### (HiPC)(OTf) (Compound 6)

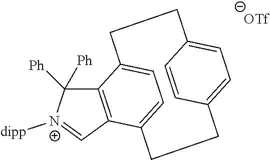

At −80° C., 5.40 g (11.4 mmol, 1.0 eq) of rac-4-bromo-5-formyl-2.2]paracyclophane-N-(2,6-diisopropylphenyl)methanimin in 15 ml of diethyl ether, 4.8 ml (2.5 M, 12 mmol, 1.05 eq) of n-butyllithium was slowly added. The red solution was stirred for 1 h at −80° C. At −80° C., a solution of 2.20 g (12.1 mmol, 1.05 eq) of benzophenone in 10 ml Et₂O was added, causing the solution to change colour from green to violet. The mixture was stirred for a further 30 min at −80° C. and then for 30 min at RT. To the yellow-orange solution, 2.0 ml (12 mmol, 1.05 eq.) of trifluoromethanesulfonic anhydride was added at −80° C. and then warmed to RT overnight. The yellow precipitate was filtered off and washed three times with 5 ml diethyl ether. The product was obtained by subliming diethyl ether into a THF solution as yellow crystals (3.60 g, 5.07 mmol, 45%).

The spectroscopic data are as follows and shown in FIGS. 4 (¹H-NMR) and 5 (¹³C-NMR):

¹H-NMR (600 MHz, CD₂Cl₂) δ 9.65 (s, 1H), 8.10 (bs, 1H), 7.71 (bs, 1H), 7.56 (m, 1H), 7.45 (bs, 1H), 7.37 (m, 1H), 7.33 (m, 1H), 7.30 (m, 1H), 7.17 (bs, 1H), 7.05 (bs, 1H), 6.93 (m, 1H), 6.85 (m, 1H), 6.82 (m, 1H), 6.80 (m, 1H), 6.78 (m, 1H), 6.61 (m, 1H), 5.29 (m, 1H), 3.99 (m, 1H), 3.75 (m, 1H), 3.56 (m, 1H), 3.31 (sept, J=6.7 Hz, 1H), 3.17 (m, 1H), 2.97 (m, 1H), 2.78 (m, 1H), 2.60 (m, 1H), 2.40 (m, 1H), 1.31 (d, J=6.7 Hz, 3H), 0.86 (sept., J=6.7 Hz, 1H), 0.70 (d, J=6.8 Hz, 3H), 0.49 (d, J=6.7 Hz, 3H), −0.12 (d, J=6.6 Hz, 3H).

¹³C-NMR (151 MHz, CD₂Cl₂) δ 174.0 (s), 148.6 (s), 147.2 (s), 146.8 (s), 146.7 (s), 143.3 (s), 140.6 (s), 138.9 (s), 137.9 (s), 137.1 (s), 133.6 (s), 133.0 (s), 132.7 (s), 132.0 (s), 131.9 (s), 131.6 (s), 131.2 (s), 130.8 (s), 130.54 (d, J=1.5 Hz), 130.2 (s), 130.1 (s), 128.8 (s), 126.6 (s), 124.8 (s), 95.7 (s), 35.1 (s), 34.5 (s), 32.8 (s), 31.1 (s), 29.8 (s), 26.2 (s), 25.4 (s), 22.9 (s), 21.7 (s).

¹⁵N-NMR (60.8 MHz, CD₂Cl₂) δ −163.4.

¹⁹F-NMR (565 MHz, THF) δ −78.55.

Elemental analysis: Calculated: C, 72.76; H, 5.96; N, 1.97; Measured: C, 72.8; H, 6.2; N, 1.9.

### Examples of the Synthesis of Various Copper Complexes that can be Used as Emitter Material

### [CuCl(iPC)] (Compound 7)

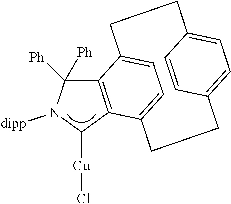

At −85° C., to a suspension of 300 mg (423 μmol, 1.0 eq.) [iPCH](OTf) and 70 mg copper(I) chloride dimethyl sulphide (0.44 mmol, 1.05 eq.) in 20 ml THF, a solution of 69 mg (0.43 mmol, 1.0 eq) KHMDS in 2 mL THF was slowly added. The yellow-orange suspension was warmed to RT overnight. All volatile components of the yellow-green suspension were removed under reduced pressure. After extraction with DCM and filtration over basic aluminium oxide, the crude product was precipitated by adding pentane and washed twice with 2 ml pentane. The product was obtained as orange crystals by sublimation of pentane into a THF/cyclohexane solution as a 1:1 adduct with THF (112 mg, 170 μmol, 40%).

The spectroscopic data are as follows and shown in FIGS. 6 (¹H-NMR) and 7 (¹³C-NMR):

¹H-NMR (600 MHz, THF) δ 7.82 (m, 1H), 7.67 (m, 1H), 7.43 (m, 1H), 7.34 (m, 1H), 7.24 (m, 3H), 7.11 (bs, 2H), 6.95 (m, 1H), 6.90 (m, 1H), 6.79 (m, 1H), 6.74 (m, 1H), 6.67 (m, 1H), 6.58 (m, 1H), 6.38 (m, 1H), 5.00 (m, 1H), 4.81 (m, 1H), 3.54 (sept., J=6.7 Hz, 1H), 3.48 (m, 1H), 3.23 (m, 1H), 2.94 (m, 1H), 2.71 (m, 1H), 2.54 (m, 1H), 2.41 (m, 1H), 1.35 (d, J=6.8 Hz, 3H), 1.10 (sept., J=6.8 Hz, 1H), 0.81 (d, J=6.7 Hz, 3H), 0.49 (d, J=6.7 Hz, 3H), −0.20 (d, J=6.7 Hz, 3H).

¹³C-NMR (151 MHz, THF) δ 229.4 (s), 149.6 (s), 145.9 (s), 144.8 (s), 143.1 (s), 143.1 (s), 140.4 (s), 139.6 (s), 139.3 (s), 138.9 (s), 136.4 (s), 136.1 (s), 134.7 (s), 133.5 (s), 132.9 (s), 132.8 (s), 132.7 (s), 132.6 (s), 131.6 (s), 131.0 (s), 130.3 (s), 129.5 (s), 129.2 (s), 129.1 (s), 128.3 (s), 125.9 (s), 124.4 (s), 95.8 (s), 35.6 (s), 34.8 (s), 34.4 (s), 30.6 (s), 30.3 (s), 29.9 (s), 27.1 (s), 23.2 (s), 21.3 (s).

Elemental analysis: Calculated: C, 75.17; H, 6.97; N, 1.83; Measured: C, 75.1; H, 6.9; N, 1.9.

### [CuBr(iPC)] (Compound 8)

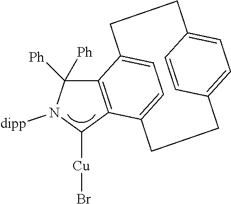

At −85° C., to a suspension of 300 mg (423 μmol, 1.0 eq) [iPCH](OTf) and 87 mg copper(I) bromide dimethyl sulfide (0.42 mmol, 1.05 eq.) in 20 mL THF, a solution of 69 mg (0.43 mmol, 1.0 eq) KHMDS in 2 mL THF was slowly added. The yellow-orange suspension was warmed to RT overnight. All volatile components of the yellow-orange suspension were removed under reduced pressure. After extraction with DCM and filtration over basic aluminium oxide, the crude product was precipitated by adding pentane and washed twice with 2 ml pentane. The product was obtained as orange crystals by sublimation of pentane into a THF/cyclohexane solution (21 mg, 30 μmol, 7%).

The spectroscopic data are as follows:

¹H-NMR (600 MHz, THF) δ 7.82 (m, 1H), 7.67 (m, 1H), 7.43 (m, 1H), 7.34 (m, 1H), 7.24 (m, 3H), 7.11 (bs, 2H), 6.95 (m, 1H), 6.90 (m, 1H), 6.79 (m, 1H), 6.74 (m, 1H), 6.67 (m, 1H), 6.58 (m, 1H), 6.38 (m, 1H), 5.00 (m, 1H), 4.81 (m, 1H), 3.54 (sept., J=6.7 Hz, 1H), 3.48 (m, 1H), 3.23 (m, 1H), 2.94 (m, 1H), 2.71 (m, 1H), 2.54 (m, 1H), 2.41 (m, 1H), 1.35 (d, J=6.8 Hz, 3H), 1.10 (sept., J=6.8 Hz, 1H), 0.81 (d, J=6.7 Hz, 3H), 0.49 (d, J=6.7 Hz, 3H), −0.20 (d, J=6.7 Hz, 3H).

Elemental analysis: Calculated: C, 71.73; H, 5.88; N, 1.99; Measured: C, 71.4; H, 6.0; N, 1.8.

### [Cu(Cbz)(iPC)] (Compound 9)

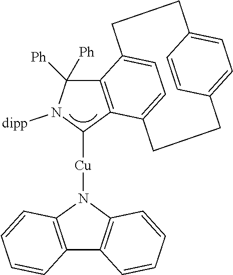

In a 20 mL screw-cap vial, 16 mg (78 μmol, 1.03 eq) of KCbz was added to a solution of 50 mg (76 μmol, 1.0 eq) of [CuCl(iPC)] in 4 mL THF and stirred overnight. The dark red suspension was mixed with 2 ml diethyl ether, filtered over basic aluminium oxide and evaporated to a quarter of the volume under reduced pressure. The product was obtained as a 2:3 adduct with THF as yellow crystals (45 mg, 57 μmol, 75%) by subliming a mixture of cyclohexane and pentane into the solution.

The spectroscopic data are as follows and shown in FIGS. 8 (¹H-NMR) and 9 (¹³C-NMR):

¹H-NMR (600 MHz, THF) δ 7.96 m, 1H), 7.83 m, 2H), 7.67 (m, 1H), 7.49 (m, 1H), 7.45 (m, 1H), 7.38 (m, 2H), 7.29 (m, 1H), 7.17 (bs, 21H), 7.01 (m, 1H), 6.97 (m, 4H), 6.80 (m, 3H), 6.73 (m, 3H), 6.62 (m, 1H), 6.50 (m, 1H), 5.13 (m, 2H), 3.90 (m, 1H), 3.72 (m, 1H), 3.64 (sept., J=6.8 Hz, 1H), 3.37 (m, 1H), 2.98 (m, 1H), 2.74 (m, 1H), 2.61 (m, 1H), 2.43 (m, 1H), 1.26 (m, 1H), 1.13 (d, J=6.7 Hz, 3H), 0.87 (d, J=6.8 Hz, 3H), 0.51 (d, J=6.7 Hz, 3H), −0.16 (d, J=6.7 Hz, 3H).

¹³C-NMR (151 MHz, THF) δ 230.9 (s), 151.0 (s), 150.3 (s), 146.4 (s), 144.7 (s), 143.3 (s), 143.3 (s), 140.2 (s), 139.9 (s), 139.4 (s), 136.4 (s), 136.3 (s), 134.9 (s), 133.6 (s), 133.1 (s), 132.9 (s), 132.8 (s), 132.6 (s), 131.6 (s), 131.1 (s), 130.5 (s), 129.6 (s), 129.3 (s), 129.2 (s), 128.2 (s), 126.5 (s), 125.4 (s), 125.1 (s), 123.5 (s), 119.4 (s), 115.6 (s), 115.0 (s), 96.0 (s), 35.7 (s), 35.1 (s), 30.8 (s), 30.4 (s), 30.1 (s), 26.8 (s), 26.2 (s), 25.5 (s), 23.2 (s), 21.7 (s).

Elemental analysis: Calculated: C, 80.28; H, 6.85; N, 3.12; Measured: C, 79.9; H, 6.9; N, 3.3.

### [Cu(Cbz^{tBu})(iPC)] (Compound 10)

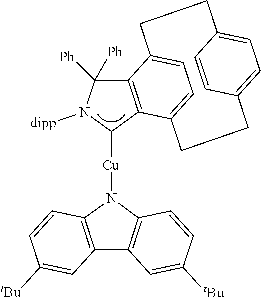

In a 20 mL screw-cap vial, 28 mg (77 μmol, 1 eq.) [KCbz^{tBu}·(Et2O)_{2/3}] were added to a solution of 50 mg (76 μmol, 1.0 eq.) [CuCl (iPC)] in 4 mL THF and stirred overnight. The dark red suspension was mixed with 2 ml diethyl ether, filtered over basic aluminium oxide and evaporated to a quarter of the volume under reduced pressure. The product was obtained as a 4:5 adduct with THE as yellow crystals (51 mg, 57 μmol, 75%) by sublimation of a mixture of cyclohexane and pentane into the solution.

The spectroscopic data are as follows and shown in FIGS. 10 (¹H-NMR) and 11 (¹³C-NMR):

¹H-NMR (600 MHz, THF) δ 7.94 (m, 1H), 7.91 (m, 2H), 7.66 (m, 1H), 7.50 (m, 1H), 7.44 (m, 1H), 7.39 (m, 1H), 7.36 (m, 1H), 7.28 (m, 1H), 7.16 (bs, 2H), 7.08 (m, 2H), 6.99 (m, 1H), 6.97 (m, 1H), 6.91 (m, 1H), 6.76 (m, 1H), 6.68 (m, 2H), 6.61 (m, 1H), 6.53 (m, 1H), 5.12 (m, 1H), 5.09 (m, 1H), 3.82 (m, 1H), 3.67 (m, 1H), 3.29 (m, 1H), 2.94 (m, 1H), 2.70 (m, 1H), 2.58 (m, 1H), 2.41 (m, 1H), 1.41 (s, 18H), 1.26 (m, 1H), 1.12 (d, J=6.7 Hz, 3H), 0.84 (d, J=6.7 Hz, 3H), 0.50 (d, J=6.7 Hz, 3H), −0.17 (d, J=6.7 Hz, 3H).

¹³C-NMR (151 MHz, THF) δ 231.0 (s), 150.3 (s), 149.6 (s), 146.3 (s), 144.7 (s), 143.3 (s), 143.2 (s), 140.2 (s), 139.8 (s), 139.8 (s), 139.4 (s), 137.6 (s), 136.4 (s), 136.2 (s), 135.0 (s), 133.7 (s), 133.0 (s), 132.9 (s), 132.8 (s), 132.6 (s), 131.5 (s), 131.0 (s), 130.4 (s), 129.5 (s), 129.3 (s), 129.1 (s), 128.2 (s), 126.4 (s), 125.3 (s), 125.0 (s), 121.1 (s), 115.3 (s), 114.5 (s), 95.9 (s), 35.6 (s), 35.0 (s), 34.9 (s), 32.6 (s), 30.8 (s), 30.4 (s), 30.1 (s), 26.9 (s), 23.2 (s), 21.7 (s).

Elemental analysis: Calculated: 81.13; H, 7.62; N, 2.82; Measured: C, 81.0; H, 7.3; N, 3.1.

### [Cu(^{Me}Cbz)(iPC)] (Compound 11)

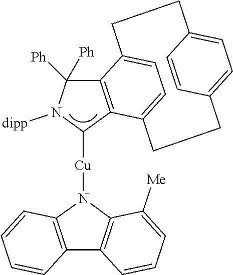

In a 20 mL screw-cap vial, 17 mg (78 μmol, 1 eq) K^{Me}Cbz was added to a solution of 50 mg (76 μmol, 1.0 eq.) [CuCl (iPC)] in 4 ml THF and stirred overnight. The dark red suspension was mixed with 2 ml diethyl ether, filtered over basic aluminium oxide and evaporated to a quarter of the volume under reduced pressure. The product was obtained as a 2:3 adduct with THE as yellow crystals (48 mg, 60 μmol, 79%) by subliming a mixture of cyclohexane and pentane into the solution.

The spectroscopic data are as follows and are shown in FIGS. 12 (¹H-NMR) and 13 (¹³C-NMR):

¹H-NMR (600 MHz, THF) δ 7.95 (m, 1H), 7.87 (m, 1H), 7.78 (m, 1H), 7.74 (m, 1H), 7.60 (bs, 1H), 7.48 (m, 1H), 7.39 (m, 1H), 7.29 (m, 3H), 7.22 (m, 2H), 7.02 (m, 3H), 6.93 (m, 1H), 6.90 (m, 1H), 6.86 (m, 1H), 6.82 (m, 1H), 6.79 (m, 1H), 6.78 (m, 1H), 6.69 (m, 1H), 6.61 (m, 1H), 6.40 (m, 1H), 5.06 (m, 1H), 4.97 (m, 1H), 3.64 (sept., J=6.7 Hz, 1H), 3.60 (m, 1H), 3.53 (m, 1H), 3.18 (m, 1H), 2.99 (m, 1H), 2.74 (m, 1H), 2.65 (m, 1H), 2.61 (s, 3H), 2.42 (m, 1H), 1.25 (m, 1H), 1.09 (d, J=6.8 Hz, 3H), 0.94 (d, J=6.7 Hz, 3H), 0.47 (d, J=6.7 Hz, 3H).

¹³C-NMR (151 MHz, THF) δ 231.4 (s), 151.2 (s), 149.8 (s), 149.6 (s), 145.9 (s), 144.8 (s), 143.5 (s), 143.0 (s), 140.3 (s), 140.0 (s), 139.7 (s), 139.5 (s), 136.5 (s), 136.4 (s), 135.1 (s), 133.7 (s), 133.0 (s), 132.7 (s), 132.7 (s), 132.5 (s), 131.8 (s), 131.0 (s), 130.6 (s), 129.6 (s), 129.4 (s), 129.3 (s), 128.2 (s), 126.3 (s), 125.9 (s), 125.3 (s), 124.8 (s), 124.6 (s), 123.2 (s), 121.5 (s), 119.5 (s), 117.6 (s), 115.8 (s), 115.8 (s), 115.7 (s), 96.1 (s), 35.7 (s), 34.6 (s), 30.8 (s), 30.5 (s), 30.2 (s), 27.0 (s), 26.2 (s), 23.5 (s), 21.7 (s), 20.9 (s).

Elemental analysis: Calculated: C, 80.36; H, 6.97; N, 3.07; Measured: C, 80.4; H, 6.9; N, 3.3.

### [Cu(^{OMe}Cbz)(iPC)] (Compound 12)

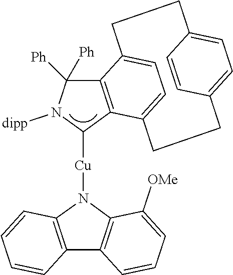

In a 20 mL screw-cap vial, 20 mg (76 μmol, 1 eq) of [K^{OMe}CBz (Et2O)_{1/3}] were added to a solution of 50 mg (76 μmol, 1.0 eq) of [CuCl(iPC)] in 4 mL THF and stirred overnight. The dark red suspension was mixed with 2 ml diethyl ether, filtered over basic aluminium oxide and evaporated to a quarter of the volume under reduced pressure. The product was obtained as a 2:3 adduct with THE as yellow crystals (46 mg, 56 μmol, 74%) by subliming a mixture of cyclohexane and pentane into the solution.

The spectroscopic data are as follows and are shown in FIGS. 14 (¹H-NMR) and 15 (¹³C-NMR):

¹H-NMR (600 MHz, THF) δ 8.01 (m, 1H), 7.81 (m, 1H), 7.73 (m, 1H), 7.55 (m, 1H), 7.47 (m, 1H), 7.38 (m, 1H), 7.28 (m, 1H), 7.26 (m, 1H), 7.24 (d, J=2.8 Hz, 1H), 7.23 (m, 1H), 7.01 (m, 1H), 6.89 (m, 2H), 6.84 (m, 1H), 6.80 (m, 3H), 6.75 (m, 2H), 6.68 (m, 1H), 6.59 (m, 1H), 6.40 (m, 1H), 5.03 (m, 1H), 4.94 (m, 1H), 4.05 (s, 3H), 3.71 (sept., J=6.7 Hz, 1H), 3.55 (m, 1H), 3.45 (m, 1H), 3.14 (m, 1H), 2.98 (m, 1H), 2.73 (m, 1H), 2.63 (m, 1H), 2.45 (m, 1H), 1.30 (sept., J=6.7 Hz, 1H), 1.28 (d, J=6.8 Hz, 3H), 0.98 (d, J=6.7 Hz, 3H), 0.52 (d, J=6.7 Hz, 3H), −0.13 (d, J=6.7 Hz, 3H).

¹³C-NMR (151 MHz, THF) δ 232.0 (s), 150.5 (s), 149.8 (s), 148.2 (s), 146.0 (s), 145.4 (s), 143.8 (s), 143.2 (s), 141.1 (s), 140.4 (s), 139.6 (s), 139.5 (s), 139.4 (s), 136.4 (s), 135.9 (s), 135.2 (s), 133.7 (s), 132.9 (s), 132.7 (s), 132.6 (s), 131.7 (s), 130.9 (s), 130.3 (s), 129.5 (s), 129.2 (s), 128.1 (s), 126.6 (s), 126.0 (s), 125.5 (s), 124.6 (s), 123.1 (s), 119.4 (s), 116.2 (s), 115.5 (s), 115.4 (s), 113.1 (s), 103.7 (s), 96.0 (s), 55.5 (s), 35.8 (s), 34.9 (s), 34.8 (s), 30.8 (s), 30.5 (s), 30.1 (s), 26.9 (s), 23.4 (s), 21.7 (s).

Elemental analysis: Calculated: C, 79.47; H, 6.67; N, 3.14; Measured: C, 79.4; H, 6.3; N, 3.4

The photophysical properties of the compounds according to the invention are shown below. UV-Vis spectra were recorded on a PerkinElmer device, model LAMBDA™ 265. Lifetime determinations, recordings of excitation and emission spectra were carried out with a FLSP920 spectrometer from Edinburgh Instruments. The emission radiation was recorded at a 90° angle to the excitation radiation. Quantum yields were determined using an integrating sphere. Quantum yields were recorded using an FLSP920 spectrometer from Edinburgh Instruments. All measurements were carried out in 1 cm quartz cuvettes.

FIG. 16 shows normalised excitation and emission spectra of compounds 6, 7 and 8, with the corresponding curves labelled with the numbers of the respective compounds. Normalised excitation and emission spectra in the ground solid (line) or in PMMA matrix (dashes) are shown. The band in the emission spectra at 425 nm is due to scattering of the light used to excite the sample.

Selected photophysical data for the compounds 6, 7 and 8 under argon at room temperature are also shown in Table 1.

TABLE 1

| | | λₘₐₓ | τ | | kᵣ |
|---|---|---|---|---|---|
| | Medium | [nm] | [μs]^{a} | φ | [10⁵ s⁻¹]^{b} |
| 6 | Solid | 520 | 0.0027 (36.3)/0.0071 (63.7) | 0.32 | 577 |
| | THF | 585 | 0.0063 | 0.01 | 31 |
| 7 | Solid | 650 | 2.8 (38.7)/4.8 (61.3) | 0.13 | 0.31 |
| | PMMA | 510 | 13 (50.0)/50 | 0.03 | 0.0062 |
| | | | (42.3)/159 (8.7) | | |
| 8 | Solid | 635 | 1.2 (40.4)/4.4 | 0.03 | 0.064 |
| | | | (42.6)/9.9 (16.9) | | |
| | PMMA | 510 | 14 (78.7)/73 (21.3) | 0.02 | 0.0089 |

^{a}For lifetimes that were fitted with two exponentials, the pre-exponential factors B are given in brackets.
^{b}kᵣ was calculated using averaged lifetimes τₐᵥ weighted according to amplitude.

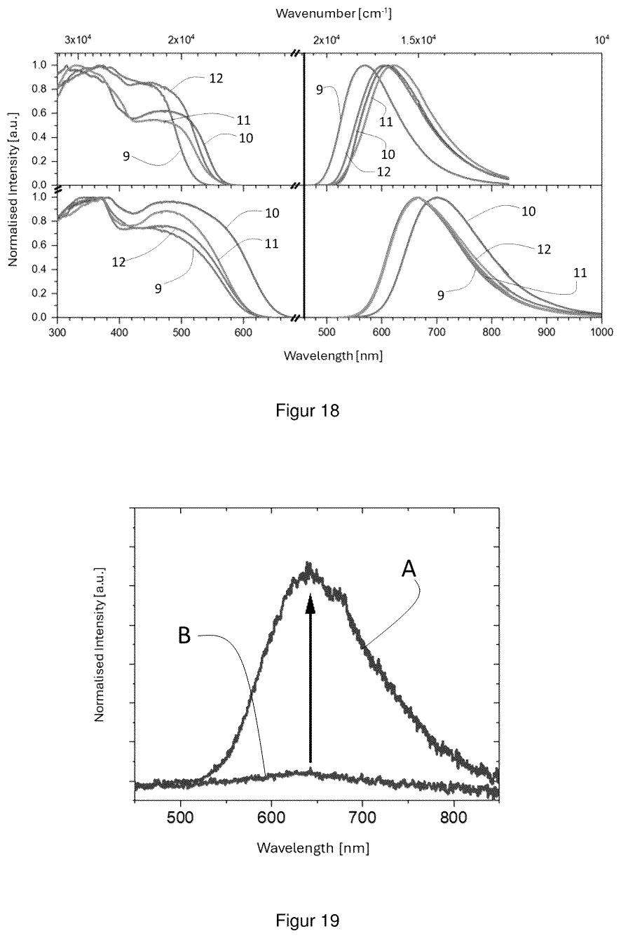

FIG. 17 shows normalised excitation (left) and emission (right) spectra of compounds 9, 10, 11 and 12 in THE solution (bottom) or PMMA matrix (top), wherein the corresponding curves are labelled with the numbers of the respective compound.

FIG. 18 shows normalised excitation (left) and emission (right) spectra of compounds 9, 10, 11 and 12 in a ground (bottom) or microcrystalline state (top), wherein the corresponding curves are labelled with the numbers of the respective compound.

Selected photophysical data for compounds 9, 10, 11 and 12 under argon at room temperature and under helium at 77 K and under vacuum at 6 K are also shown in Table 2.

TABLE 2

| | | λₘₐₓ | τ | | kᵣ |
|---|---|---|---|---|---|
| | Medium (τ) | [nm] | [μs]^{a} | φ | [10⁵ s⁻¹]^{b} |
| 9 | Microcrystalline | 570 | 0.46 (62.1)/0.77 (37.9) | 0.80 | 14 |
| | Ground | 665 | 0.18 (66.3)/0.33 (33.7) | 0.22 | 9.8 |
| | THF | 725 | 0.021 | 0.01 | 4.8 |
| | PMMA | 595 | 0.56 (59.4)/0.96 (40.6) | 0.58 | 8.1 |
| 10 | Microcrystalline (RT) | 610 | 0.39 (97.5)/0.72 (2.5) | 0.75 | 19 |
| | Microcrystalline (77K) | 595 | 9.0 (30.1)/29 | 0.95 | 0.34 |
| | | | (62.3)/89 (7.6) | | |
| | Microcrystalline (7K) | | 38 (33.3)/297 | | |
| | | | (51.7)/594 (15.0) | | |
| | Ground (RT) | 700 | 0.09 (94.6)/0.49 (5.4) | 0.11 | 9.6 |
| | Ground (77K) | 690 | 2.6 (52.6)/13 | 0.18 | 0.15 |
| | | | (35.9)/52 (11.5) | | |
| | Ground (5K) | 680 | 33 (35.0)/109 (33.9)/329 | | |
| | | | (25.7)/841 (5.4) | | |
| | THF | 785 | n.d. | n.d. | |
| | PMMA | 630 | 0.27 (43.4)/0.59 (56.6) | 0.30 | 6.6 |
| 11 | Microcrystalline | 620 | 0.18 (39.5)/0.50 (60.5) | 0.51 | 14 |
| | Ground | 665 | 0.15 (92.4)/0.48 (7.6) | 0.15 | 8.4 |
| | THF | 755 | n.d. | n.d. | |
| | PMMA | 610 | 0.35 (52.2)/0.80 (47.8) | 0.33 | 5.8 |
| 12 | Microcrystalline | 605 | 0.17 (70.5)/0.45 (29.5) | 0.29 | 11 |
| | Ground | 665 | 0.034 (83.2)/0.078 (16.8) | 0.03 | 8.0 |
| | THF | 765 | n.d. | n.d. | |
| | PMMA | 610 | 0.12 (64.4)/0.40 | 0.11 | 4.3 |
| | | | (30.9)/0.96 (4.6) | | |

^{a}For lifetimes that were fitted with two exponentials, the pre-exponential factors B are given in parentheses.
^{b}kᵣ was calculated using averaged lifetimes τₐᵥ weighted by amplitude.
n.d. = not detected.

λₘₐₓ indicates the maximum of the photoluminescence (PL) and is thus an important parameter for the resulting colour appearing in the eye. The ones found here are in the near infrared at up to 785 nm, which makes them interesting for various luminescence applications.

The quantum yield φ_{PL} is a measure of the efficiency of a luminophore. It indicates the proportion of excited states that radiatively transition to the ground state and can be understood as the ratio of emitted to absorbed photons. Although the φ_{PL} given here refers to PL, this value is also an interesting measure of the efficiency of electroluminescence within an electrically operated component. The lifetime τ is a measure of how quickly the luminophores return to their ground state after excitation (whereby they emit a photon at φ_{PL}=1). T should be as small as possible to avoid ‘efficiency roll-off’ effects in OLEDs. The radiative rate constant k_{R}=φ_{PL}/τ can be calculated from φ_{PL} and τ. This can be used to compare the complexes with other emitters. The highest value determined here of k_{R}=19×10⁵ s⁻¹ already exceeds many currently used Ir or Pt emitters. Generally, there are hardly any emitter materials that have such a high radiative rate constant.

In the following, chiroptical properties of compounds according to the disclosure are described.

The anisotropy factors g_{abs} and gₗᵤₘ are a measure of how much left- and right-turning photons dominate over the others during absorption and emission, respectively. The following applies:

$$\text{?}$$

$$\text{?} \text{indicates text missing or illegible when filed}$$

where I_{L} and I_{R} represent the intensities of left- and right-turning photons, respectively. The maxima +2 for pure left-turning and −2 for pure right-turning emitted light result from the equation. For octahedral transition metal complexes (such as Ir(III)) and comparable known copper complexes, these values are rarely above 10⁻³.

Analogously,

$$\text{?}$$

$$\text{?} \text{indicates text missing or illegible when filed}$$

from the extinction coefficients ϵ_{L} and ϵ_{R} for the respective absorbed photons. g_{abs} is actually less important for us. An anisotropy factor g_{abs}≈2×10⁻³ was calculated for the lowest-energy absorption. The g_{abs} of the energetically lowest band should be in the same order of magnitude as the gₗᵤₘ value, since the same electronic states should be involved in a TADF emission mechanism as in the lowest-energy absorption (see Einstein coefficient). According to the disclosure, a value of gₗᵤₘ≈2×10⁻³ is expected. This is then quite comparable with those of phosphorescent or TADF emitters.

Regarding the application in organic light-emitting diodes, the following should be noted.

By integrating a molecular emitter into an organic light-emitting diode (OLED), it is possible to electrically excite the molecule by means of an electrical field, analogous to optical excitation (PL), which is referred to as electroluminescence (EL). OLEDs are particularly relevant for display/screen applications. The described TADF emission behaviour of the emitters according to the disclosure, combined with high quantum yields φ_{PL}, makes these materials particularly interesting for use in OLED components, as they can theoretically achieve high external quantum efficiencies (EQE). The EQE is defined by the number of photons that are coupled out of the OLED per injected charge carrier. An important influencing factor is the internal quantum efficiency (IQE), which can reach a maximum of 100% for phosphorescent and TADF emitters.

To test the electroluminescent operation of the iPC emitter, compound 10 was integrated into a simple proof-of-concept OLED. The device architecture can be described in conjunction with FIG. 1. Indium tin oxide (ITO) acts as a transparent anode 14 (hole-injecting contact), PEDOT:PSS as a hole injection/transport material and calcium/aluminium as an electron-injecting top contact. The emitter is embedded in a matrix material (mCP) via solution processing in an amount of 10 wt. %, based on the layer. This emitting layer (consisting of an emitter and matrix) is located between the PEDOT:PSS layer and the top contact. When bipolar charge carriers are injected into the emitting layer, excitons (excited states) are generated on the emitter, which can undergo radiative decay (EL) and result in light being coupled out via the transparent ITO anode.

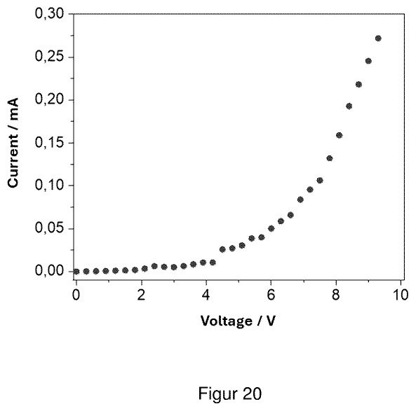

FIG. 19 also shows that the measured EL spectra correspond to the PL spectra in terms of spectral position and shape, which shows that the emitter according to the disclosure can be electrically driven in OLED components according to compound 10. The current density here is of an order of magnitude typical for OLEDs. The intensity of the EL scales as expected with the increase in current density. Here, curve A shows a current density j of 33 mA/cm² and curve B shows a current density of 17 mA/cm².

FIG. 20 shows the current-voltage characteristic at positive voltage (forward direction) and shows typical diode behaviour. It can be seen that the current flow in the OLED is dominated by injection via the contact interfaces.

As used herein, the terms “general,” “generally,” and “approximately” are intended to account for the inherent degree of variance and imprecision that is often attributed to, and often accompanies, any design and manufacturing process, including engineering tolerances, and without deviation from the relevant functionality and intended outcome, such that mathematical precision and exactitude is not implied and, in some instances, is not possible.

All the features and advantages, including structural details, spatial arrangements and method steps, which follow from the claims, the description and the drawing can be fundamental to the invention both on their own and in different combinations. It is to be understood that the foregoing is a description of one or more preferred exemplary embodiments of the invention. The invention is not limited to the particular embodiment(s) disclosed herein, but rather is defined solely by the claims below. Furthermore, the statements contained in the foregoing description relate to particular embodiments and are not to be construed as limitations on the scope of the invention or on the definition of terms used in the claims, except where a term or phrase is expressly defined above. Various other embodiments and various changes and modifications to the disclosed embodiment(s) will become apparent to those skilled in the art. All such other embodiments, changes, and modifications are intended to come within the scope of the appended claims.

As used in this specification and claims, the terms “for example,” “for instance,” “such as,” and “like,” and the verbs “comprising,” “having,” “including,” and their other verb forms, when used in conjunction with a listing of one or more components or other items, are each to be construed as open-ended, meaning that the listing is not to be considered as excluding other, additional components or items. Other terms are to be construed using their broadest reasonable meaning unless they are used in a context that requires a different interpretation.

1. A paracyclophane-carbene compound, wherein the paracyclophane-carbene compound corresponds to the following structure (I): 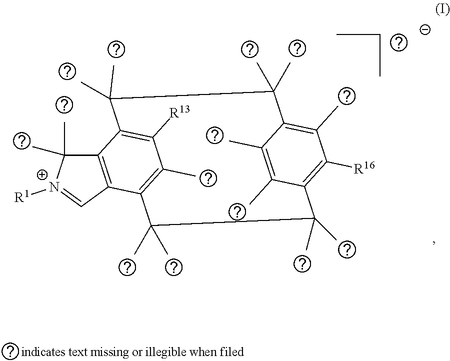
wherein
R¹ is selected from aryl, heteroaryl, alkyl, perfluoroalkyl, perfluoroaryl, wherein
R² and R³ are the same or different and are independently selected from aryl, heteroaryl, alkyl, alkenyl, alkynyl, perfluoroalkyl, perfluoroaryl, wherein
R⁴, R⁵, R⁶, R⁷, R⁸, R⁹, R¹⁰, R¹¹ are the same or different and are independently selected from hydrogen, deuterium, halogen, silyl, alkyl, alkenyl, alkynyl, perfluoroalkyl, aryl, heteroaryl, alkoxy, wherein
R¹² R¹³, R¹⁴, R¹⁵, R¹⁶, R¹⁷ are the same or different and are independently selected from hydrogen, deuterium, halogen, alkyl, alkenyl, alkynyl, perfluoroalkyl, aryl, heteroaryl, alkoxy, COR¹⁸, CO₂R¹⁹, CONR²⁰, cyano, nitro, SO₃R²¹, PO₃R²²₂, wherein
R¹⁸ R¹⁹, R²⁰, R²¹, R²² are the same or different and are independently selected from hydrogen, deuterium, halogen, silyl, alkyl, alkenyl, alkynyl, perfluoroalkyl, aryl, heteroaryl, alkoxy, and wherein
An⁻ is an anion.

2. A paracyclophane-carbene compound according to claim 1,
wherein R¹ is selected from C5 to C18 aryl, C5 to C18 heteroaryl, C1 to C18 alkyl, C1 to C18 perfluoroalkyl, C5 to C18 perfluoroaryl, wherein
R² and R³ are the same or different and are independently selected from
C5 to C18 aryl, C5 to C18 perfluoroaryl, C5 to C18 heteroaryl, C1 to C12 alkyl, C1 to C12 alkenyl, C1 to C12 alkynyl, C1 to C12 perfluoroalkyl, wherein
R⁴, R⁵, R⁶, R⁷, R⁸, R⁹, R¹⁰, R¹¹ are the same or different and are independently selected from hydrogen, deuterium, halogen, silyl, C1 to C12 alkyl, C1 to C12 alkenyl, C1 to C12 alkynyl and C1 to C12 perfluoroalkyl, C5 to C18 aryl, C5 to C18 perfluoroaryl, C5 to C18 heteroaryl, C1 to C12 alkoxy, wherein
R¹² R¹³, R¹⁴, R¹⁵, R¹⁶, R¹⁷ are the same or different and are independently selected from hydrogen, C1 to C18 alkyl, C1 to C18 alkenyl, C1 to C18 alkynyl, and C1 to C18 perfluoroalkyl, C5 to C18 aryl, C5 to C18 perfluoroaryl, C5 to C18 heteroaryl, C1 to C18 alkoxy, COR¹⁸, CO₂R¹⁹, CONR²⁰, cyano, nitro, SO₃R²¹, PO₃R²²₂, wherein
R¹⁸ R¹⁹, R²⁰, R²¹, R²² are the same or different and are independently selected from hydrogen, deuterium, halogen, silyl, C1 to C18 alkyl, C1 to C18 alkenyl, C1 to C18 alkynyl, C1 to C18 perfluoroalkyl, C5 to C18 aryl, C5 to C18 heteroaryl, C1 to C18 alkoxy, and wherein
An⁻ is an anion selected from halide, triflate, sulphate, phosphate, silicate, tetrafluoroborate, hexafluorophosphate, tetraarylborate.

3. A paracyclophane-carbene compound according to claim 1, wherein R¹ is selected from C9 to C18 aryl, C9 to C18 heteroaryl, C1 to C12 alkyl, C1 to C12 perfluoroalkyl, C9 to C18 perfluoroaryl, wherein
R² and R³ are the same or different and are independently selected from C5 to C6 aryl and C5 to C6 heteroaryl,
R⁴, R⁵, R⁶, R⁷, R⁸, R⁹, R¹⁰, R¹¹ are the same or different and are independently selected from hydrogen, deuterium, halogen and silyl, C1 to C4 alkyl, C1 to C4 alkenyl, C1 to C4 alkinyl and C1 to C4 perfluoroalkyl, C5 to C6 aryl, C5 to C6 heteroaryl, C5 to C6 perfluoroaryl, C1 to C4 alkoxy, wherein
R¹² R¹³, R¹⁴, R¹⁵, R¹⁶, R¹⁷ are the same or different and are independently selected from hydrogen, C1 to C4 alkyl, C1 to C4 alkenyl, C1 to C4 alkynyl, and C1 to C4 perfluoroalkyl, C5 to C6 aryl, C5 to C6 perfluoroaryl, C5 to C6 heteroaryl, C1 to C4 alkoxy, COR¹⁸, CO₂R¹⁹, CONR²⁰, cyano, nitro, SO₃R²¹, PO₃R²²₂, wherein
R¹⁸ R¹⁹, R²⁰, R²¹, R²² are the same or different and are independently selected from hydrogen, deuterium, halogen, silyl, C1 to C4 alkyl, C1 to C4 alkenyl, C1 to C4 alkynyl, C1 to C4 perfluoroalkyl, C5 to C12 aryl, C5 to C12 heteroaryl, C to C4 alkoxy, and wherein
An⁻ is an anion selected from halide, triflate, sulphate, phosphate, silicate, tetrafluoroborate, hexafluorophosphate, tetraarylborate.

4. A paracyclophane-carbene compound according to claim 1, wherein R¹ is dialkylphenyl.

5. A paracyclophane-carbene compound according to claim 1, wherein R² and R³ are phenyl.

6. A paracyclophane-carbene compound according to claim 1, wherein R⁴, R⁵, R⁶, R⁷, R⁸, R⁹, R¹⁰ and R¹¹ are hydrogen.

7. A paracyclophane-carbene compound according to claim 1, wherein R¹² R¹³, R¹⁴, R¹⁵, R¹⁶ and R¹⁷ are hydrogen.

8. A paracyclophane-carbene compound according to claim 1, wherein the paracyclophane carbene compound has the following structure (II):

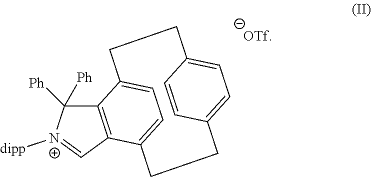

9. Use of a paracyclophane-carbene compound according to claim 1 for preparing a metal complex for an emitter material of an emitter layer of an organic light-emitting diode.

10. Metal complex, wherein the metal complex corresponds to the following structure (III): 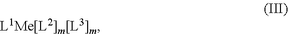
wherein L¹ is a paracyclophane-carbene ligand according to claim 1, wherein
L² and L³ are the same or different and are independently selected from
NR²³₂, NR²⁴₃, PR²⁵₂, PR²⁶₃, OR²⁷₂, OR²⁸, SR²⁹, SR³⁰₂, SR³¹, SeR³²₂, SeR³⁴, TeR³⁵₂, TeR³⁶, silyl, aryl, heteroaryl, alkenyl, alkynyl, isonitrile, cyclopentadienyl, halogen, wherein
m is a number selected from 0, 1 or 2, with the proviso that at least one m is not equal to zero, wherein
R²³, R²⁴, R²⁵, R²⁶, R²⁷, R²⁸, R²⁹, R³⁰, R³¹, R³², R³³, R³⁴, R³⁵, R³⁶ are the same or different and are independently selected from aryl, heteroaryl, alkenyl, alkynyl, alkyl, perfluoroalkyl, perfluoroaryl; and wherein
Me is a metal selected from copper, silver and gold.

11. A metal complex as claimed in claim 10, wherein
L¹ is a paracyclophane-carbene ligand, wherein
Me is a metal selected from copper, silver and gold, and wherein
L² is a ligand selected from a heteroaryl and a halogen.

12. A metal complex according to claim 10, wherein
L¹ is a paracyclophane-carbene ligand, wherein
Me is copper, and wherein
L² is a ligand selected from a carbazole ligand and a halogen.

13. A metal complex according to claim 10, wherein the metal complex is selected from the structures (IV), (V), (VI), (VII), (VIII) and (IX) as shown below:

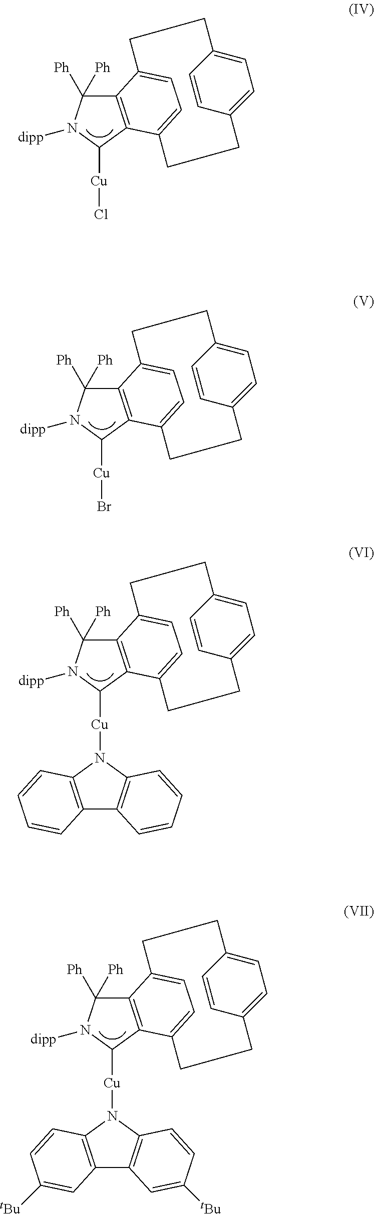

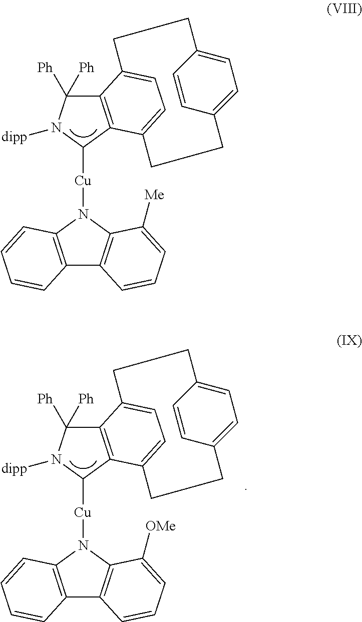

14. Use of a metal complex as the emitter material in an emitter layer of an organic light-emitting diode, wherein the metal complex is formed according to claim 10.

15. Emitter layer for an organic light-emitting diode, having a metal complex for emitting light, wherein the metal complex is formed according to claim 10.

16. Organic light-emitting diode comprising a cathode, an emitter layer, a hole transport layer and an anode, wherein the emitter layer is formed according to claim 15.

17. Organic light-emitting diode according to claim 16, wherein the organic light-emitting diode further comprises a filter for reducing reflections of external light sources, the filter being at least partially opaque to linearly polarised light.

18. A method for generating light by means of an organic light-emitting diode, comprising the steps of:
a) providing an organic light-emitting diode according to claim 16, and
b) applying a voltage between the anode and the cathode to inject charge carriers and generate light.
